(19) United States
(12) Patent Application Publication
(10) Pub. No.: US 2003/0060489 A1
(43) Pub. Date: Mar. 27, 2003

(54) Novel method of treatment

(76) Inventor: Robin Edwin Buckingham, Harlow (GB)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/168,872

(22) PCT Filed: Dec. 22, 2000
(86) PCT No.: PCT/GB00/05006

(30) Foreign Application Priority Data
Dec. 24, 1999 (GB) 9930688.8

(51) Int. Cl. A61K 31/4439; A61K 31/427; A61K 31/137

(52) U.S. Cl. 514/342; 514/369; 514/651

(57) ABSTRACT

A method for the treatment of diabetes mellitus, especially Type II diabetes and the cardiac conditions associated with diabetes mellitus in a mammal such as a human, which method comprises administering an effective non-toxic and pharmaceutically acceptable amount of an insulin sensitizer, such as Compound (I), and an agent used in the treatment of the cardiac conditions associated with diabetes mellitus.

This invention relates to a method of treatment, in particular to a method for the treatment of diabetes mellitus, especially non-insulin dependent diabetes (NIDDM) or Type II diabetes, and the cardiac conditions associated with diabetes mellitus.

It is known that a common sequela of the Type II diabetes syndrome is the development of cardiac conditions. In particular, it is known that Type II diabetes can lead to the development of heart failure, including congestive heart failure (also known as chronic heart failure).

Agents used in the treatment of heart failure, including congestive heart failure, include endothelin antagonists, beta-blockers, ACE inhibitors and diuretics.

Endothelin is a potent vasoconstrictor peptide synthesized and released by the vascular endothelium. International Patent Applications Publication Numbers WO 93/08799 and WO 94/25013 (SmithKline Beecham Corporation) disclose certain indanes and indenes as endothelin receptor antagonists. International Patent Application Publication Number WO 97/04772 (SmithKline Beecham Corporation) discloses certain pyrroles, pyrazoles, and triazoles as endothelin receptor antagonists.

Beta-blockers are compounds that act as competitive antagonists at beta-adrenergic receptor sites and include the compounds disclosed in reference texts such as Martindale 32nd Edition “The Complete Drug Reference” especially page 777. The alkanolamines are well known examples of beta-blockers. The alkanolamines are used in the treatment of cardiovascular disorders such as hypertension, angina pectoris, cardiac arrhythmias, and myocardial infarction.

Angiotensin converting enzyme (ACE) inhibitors are compounds that are used in the treatment of heart failure, hypertension, and myocardial infarction. And include the compounds disclosed in reference texts such as Martindale 32nd Edition “The Complete Drug Reference”, especially page 776.

Diuretics are compounds which promote the excretion of urine and thus a reduction in blood plasma volume and include those compounds disclosed in reference texts such as Martindale 32nd Edition The Complete Drug Reference, especially page 778. Diuretic compounds may be divided into classes according to their mode of action. These classes include carbonic anhydrase inhibitors, loop diuretics, potassium-sparing diuretics, and thiazides

European Patent Application, Publication Number 0,306,228 relates to certain thiazolidinedione derivatives disclosed as having antihyperglycaemic and antihyperlipidaemic activity. One particular thiazolidinedione disclosed in EP 0306228 is 5-[4-[2-(N-methyl-N-(2-pyridyl)amino)ethoxy]benzy]thiazolidine -2,4-dione (hereinafter ‘Compound (I)’). WO 94/05659 discloses certain salts of Compound (I) including the maleate salt at example 1 thereof.

Compound (I) is an example of a class of anti-hyperglycaemic agents known as ‘insulin sensitisers’. In particular Compound (I) is a thiazolidinedione insulin sensitiser.

European Patent Applications, Publication Numbers: 0008203, 0139421, 0032128, 0428312, 0489663,0155845, 0257781, 0208420, 0177353, 0319189, 0332331, 0332332, 0528734, 0508740; International Patent Application, Publication Numbers 92/18501, 93/02079, 93/22445 and U.S. Pat. Nos. 5,104,888 and 5478852, also disclose certain thiazolidinedione insulin sensitisers.

Another series of compounds generally recognised as having insulin sensitiser activity are those typified by the compounds disclosed in International Patent Applications, Publication Numbers WO 93/21166 and WO 94/01420. These compounds are herein referred to as ‘acyclic insulin sensitisers’. Other examples of acyclic insulin sensitisers are those disclosed in U.S. Pat. No. 5,232,945 and International Patent Applications, Publication Numbers WO 92/03425 and WO 91/19702. Further examples of insulin sensitiser are those disclosed in WO 97/31907 and GW262570.

Examples of other insulin sensitisers are those disclosed in European Patent Application, Publication Number 0533933, Japanese Patent Application Publication Number 05271204 and U.S. Pat. No. 5,264,451.

The above mentioned publications are incorporated herein by reference.

It is now considered that Compound (1) in combination with an agent used in the treatment of heart failure, including congestive heart failure, provides a beneficial effect upon glycaemic control and ameliorates the cardiac conditions associated with diabetes mellitus, especially that associated with the onset and development of heart failure, beyond a mere additive effect. Such a combination is therefore particularly useful for the treatment of diabetes mellitus, especially Type II diabetes and the cardiac conditions arising from diabetes mellitus. The treatment is also indicated to proceed with minimum side effects.

Accordingly, the invention provides a method for the treatment of diabetes mellitus, especially Type II diabetes and the cardiac conditions associated with diabetes mellitus in a mammal such as a human, which method comprises administering an effective non-toxic and pharmaceutically acceptable amount of an insulin sensitiser, such as Compound (I), and an agent used in the treatment of the cardiac conditions associated with diabetes mellitus.

Cardiac conditions associated with diabetes mellitus includes those cardiac conditions arising from diabetes mellitus.

Cardiac conditions arising from diabetes mellitus include heart failure, for example congestive heart failure.

A suitable agent used in the treatment of cardiac conditions associated with diabetes mellitus, such as heart failure, includes a beta-blocker, an ACE inhibitor or a diuretic. A suitable agent used in the treatment of cardiac conditions associated with diabetes mellitus, such as heart failure, also includes an endothelin antagonist.

In another aspect the invention provides an insulin sensitiser, such as Compound (I), and an agent used in the treatment of the cardiac conditions associated with diabetes mellitus, for use in a method for the treatment of diabetes mellitus, especially Type II diabetes, and the cardiac conditions associated with diabetes mellitus.

The method comprises either co-administration of an insulin sensitiser, such as Compound (I), and the agent used in the treatment of the cardiac conditions associated with diabetes mellitus or the sequential administration thereof.

Co-administration includes administration of a formulation which includes both an insulin sensitiser, such as Compound (I), and an agent used in the treatment of the cardiac conditions associated with diabetes mellitus or the essentially simultaneous administration of separate formulations of each agent.

In another aspect the invention provides the use of an insulin sensitiser, such as Compound (I), and an agent used in the treatment of the cardiac conditions associated with diabetes mellitus, for use in the manufacture of a composition for the treatment of diabetes mellitus, especially Type II diabetes and the cardiac conditions associated with diabetes mellitus.

A suitable thiazolidinedione insulin sensitiser is Compound (I).

Other suitable thiazolidinedione insulin sensitisers include (+)-5-[[4-[(3,4-dihydro-6-hydroxy-2,5,7,8-tetramethyl-2H-1-benzopyran-2-yl)methoxy]phenyl]methyl]-2,4-thiazolidinedione (or troglitazone), 5-[4-[(1-methylcyclohexyl)methoxy]benzyl]thiazolidine-2,4-dione (or ciglitazone), 5-[4-[2-(5-ethylpyridin-2-yl)ethoxy]benzyl]thiazolidine-2,4-dione (or pioglitazone), and 5-[(2-benzyl-2,3-dihydrobenzopyran)-5-ylmethyl)thiazolidine-2,4-dione (or englitazone).

Suitable endothelin antagonists include those disclosed in WO 93/08799, WO 94/25013, and WO 97/04772.

Suitable beta-blockers include acebutolol, alprenolol, amosulatol, arotinolol, atenolol, befinolol, betaxolol, bevantolol, bisoprolol, bopindolol, bucindolol, bufetolol, bufuralol, bunitrolol, bupranolol, carazolol, carteolol, carvedilol, celiprolol, chloranolol, dilevalol, epanolol, esmolol, flestolol, indenolol, labetalol, levobunolol, levomoprolol, medroxalol, mepindolol, metipranolol, metoprolol, nadolol, nebivolol, nipradilol, oxprenolol, penbutolol, pindolol, practolol, propranolol, sotalol, talinolol, tertatolol, tilisolol, and timolol.

Suitable ACE inhibitors include alacepril, benazepril, captopril, ceronapril, cilazepril, delapril, enalapril, enalaprilat, fosinopril, imidapril, libenzapril, lisinopril, moexipril, moveltipril, pentopril, perindopril, quinapril, ramipril, spirapril, temocapril, teprotide, trandolapril, and zofenopril.

Suitable diuretics include acetazolamide, brinzolamide, dichlorphenamide, dorzolamide, methazolamide, azosemide, bumetanide, ethacrynic acid, etozolin, frusemide, piretanide, torasemide, isosorbide, mannitol, amiloride, canrenoate potassium, canrenone, spironolactone, triamterene, althiazide, bemetizide, bendrofluazide, benzthiazide, buthiazide, chlorothiazide, chlorthalidone, clopamide, cyclopenthiazide, cyclothiazide, epithiazide, hydrochlorothiazide, hydroflumethiazide, indapamide, mebutizide, mefruside, methylclothiazide, meticrane, metolazone, polythiazide, quinethazone, teclothiazide, trichlormethiazide, tripamide, and xipamide.

In one particular aspect, the method comprises the administration of up to 12 mg such as 2 to 12 mg of Compound (I), especially when administered per day.

Particularly, the method comprises the administration of 2 to 4, 4 to 8 or 8 to 12 mg of Compound (I) per day.

Particularly, the method comprises the administration of 2 to 4 mg of Compound (I), especially when administered per day.

Particularly, the method comprises the administration of 4 to 8 mg of Compound (I), especially when administered per day.

Particularly, the method comprises the administration of 8 to 12 mg of Compound (I), especially when administered per day.

Preferably, the method comprises the administration of 2 mg of Compound (I), especially when administered per day.

Preferably, the method comprises the administration of 4 mg of Compound (I), especially when administered per day.

Preferably, the method comprises the administration of 8 mg of Compound (I), especially when administered per day.

It will be understood that the insulin sensitiser, such as Compound (I) and the agent used in the treatment of the cardiac conditions associated with diabetes mellitus such as the endothelin antagonist, the beta-blocker, the ACE inhibitor, or the diuretic are each administered in a pharmaceutically acceptable form, including pharmaceutically acceptable derivatives such as pharmaceutically acceptable salts, esters and solvates thereof, as appropriate of the relevant pharmaceutically active agent. In certain instances herein the names used for the relevant agent used in the treatment of the cardiac conditions associated with diabetes mellitus may relate to a particular pharmaceutical form of the relevant active agent. It will be understood that all pharmaceutically acceptable forms of the active agents per se are encompassed by this invention.

Suitable pharmaceutically acceptable salted forms of Compound (I) include those described in EP 0306228 and WO94/05659. A preferred pharmaceutically acceptable salt is a maleate.

Suitable pharmaceutically acceptable solvated forms of Compound (I) include those described in EP 0306228 and WO94/05659, in particular hydrates.

Suitable pharmaceutically acceptable forms of other active agents including the agent used in the treatment of the cardiac conditions associated with diabetes mellitus depend upon the particular agent used but include known pharmaceutically acceptable forms of the particular agent chosen, for example those disclosed in the above mentioned publications.

Compound (I) or, a pharmaceutically acceptable salt thereof, or a pharmaceutically acceptable solvate thereof, may be prepared using known methods, for example those disclosed in EP 0306228 and WO94/05659. The disclosures of EP 0306228 and WO94/05659 are incorporated herein by reference.

Certain of the active agents including the thiazolidinedione insulin sensitisers such as Compound (I) may exist in one of several tautomeric forms, all of which are encompassed in this invention as individual tautomeric forms or as mixtures thereof. Certain of the active agents mentioned herein, including Compound (I) contain one or more chiral carbon atom, and hence can exist in distinct stereoisomeric forms, the present invention encompasses all of these isomeric forms whether as individual isomers or as mixtures of isomers, including racemates.

The particular method of preparation of the active agent used in the invention will depend upon the agent chosen but will in general be selected from methods known in the art.

The endothelin antagonist of choice is prepared according to known methods, for example those disclosed in WO 93/08799, WO 94/25013, and WO 97/04772.

The beta-blocker of choice is prepared according to known methods, such methods are found or are referred to in standard reference texts, such as the British and US Pharmacopoeias, Remington's Pharmaceutical Sciences (Mack Publishing Co.), Martindale 32nd Edition The Complete Drug Reference (London, The Pharmaceutical Press) or the above mentioned publications.

The ACE inhibitor of choice is prepared according to known methods, such methods are found or are referred to in standard reference texts, such as the British and US Pharmacopoeias, Remington's Pharmaceutical Sciences (Mack Publishing Co.), Martindale 32nd Edition The Complete Drug Reference (London, The Pharmaceutical Press) or the above mentioned publications.

The diuretic of choice is prepared according to known methods, such methods are found or are referred to in standard reference texts, such as the British and US Pharmacopoeias, Remington's Pharmaceutical Sciences (Mack Publishing Co.), Martindale 32nd Edition The Complete Drug Reference (London, The Pharmaceutical Press) or the above mentioned publications.

As used herein the term ‘pharmaceutically acceptable’ embraces both human and veterinary use: for example the term ‘pharmaceutically acceptable’ embraces a veterinarily acceptable compound.

For the avoidance of doubt, when reference is made herein to scalar amounts, including mg amounts, of Compound (I) in a pharmaceutically acceptable form, the scalar amount referred to is made in respect of Compound (I) per se. For example 2 mg of Compound (I) in the form of the maleate salt is that amount of maleate salt which contains 2 mg of Compound (I).

Diabetes mellitus is preferably Type II diabetes.

The term ‘cardiac conditions’ includes heart failure.

Glycaemic control may be characterised using conventional methods, for example by measurement of a typically used index of glycaemic control such as fasting plasma glucose or glycosylated haemoglobin (Hb Alc). Such indices are determined using standard methodology, for example those described in: Tuescher A, Richterich, P., Schweiz. med. Wschr. 101 (1971), 345 and 390 and Frank P., ‘Monitoring the Diabetic Patent with Glycosolated Hemoglobin Measurements’, Clinical Products 1988.

In the method of the invention, the active medicaments are preferably administered in pharmaceutical composition form. As indicated above, such compositions can include both medicaments or one only of the medicaments. Accordingly, in one aspect the present invention also provides a pharmaceutical composition comprising an insulin sensitiser, such as Compound (I) especially 2 to 12 mg thereof, an agent used in the treatment of the cardiac conditions associated with diabetes mellitus and a pharmaceutically acceptable carrier therefor.

Such compositions may be prepared by admixing an insulin sensitiser, such as Compound (I) especially 2 to 12 mg thereof, the agent used in the treatment of the cardiac conditions associated with diabetes mellitus and a pharmaceutically acceptable carrier therefor.

Usually the compositions are adapted for oral administration. However, they may be adapted for other modes of administration, for example parenteral administration, sublingual or transdermal administration.

The compositions may be in the form of tablets, capsules, powders, granules, lozenges, suppositories, reconstitutable powders, or liquid preparations, such as oral or sterile parenteral solutions or suspensions.

In order to obtain consistency of administration it is preferred that a composition of the invention is in the form of a unit dose.

Unit dose presentation forms for oral administration may be tablets and capsules and may contain conventional excipients such as binding agents, for example syrup, acacia, gelatin, sorbitol, tragacanth, or polyvinylpyrrolidone; fillers, for example lactose, sugar, maize starch, calcium phosphate, sorbitol or glycine; tabletting lubricants, for example magnesium stearate; disintegrants, for example starch, polyvinylpyrrolidone, sodium starch glycollate or microcrystalline cellulose; or pharmaceutically acceptable wetting agents such as sodium lauryl sulphate.

The compositions are preferably in a unit dosage form in an amount appropriate for the relevant daily dosage.

Suitable dosages including unit dosages of the Compound of formula (I) comprise 1, 2, 3, 4, 5, 6, 7, 8, 9, 10, 11 or 12 mg of Compound (I).

In the treatment the medicaments may be administered from 1 to 6 times a day, but most preferably 1 or 2 times per day.

Particular dosages of Compound (I) are 2 mg/day, 4 mg/day, including 2 mg twice per day, and 8 mg/day, including 4 mg twice per day.

Suitable dosages of the agent used in the treatment of the cardiac conditions associated with diabetes mellitus are those used in the art for the particular agent chosen.

Suitable dosages of the endothelin antagonist depend on the endothelin antagonist chosen, but include those disclosed in WO 93/08799, WO 94/25013, and WO 97/04772.

Suitable dosages of the beta-blocker, such as the alkanolamine, include the known dosages, including unit doses, for these compounds as described or referred to in reference text such such as the British and US Pharmacopoeias, Remington's Pharmaceutical Sciences (Mack Publishing Co.), Martindale 32nd Edition The Complete Drug Reference (London, The Pharmaceutical Press), or the above mentioned publications.

Suitable dosages of the ACE inhibitor include the known dosages, including unit doses, for these compounds as described or referred to in reference text such as the British and US Pharmacopoeias, Remington's Pharmaceutical Sciences (Mack Publishing Co.), Martindale 32nd Edition The Complete Drug Reference (London, The Pharmaceutical Press) or the above mentioned publications.

Suitable dosages including unit dosages of the diuretic include the known dosages including unit doses for these compounds as described or referred to in reference text such as the British and US Pharmacopoeias, Remington's Pharmaceutical Sciences (Mack Publishing Co.), Martindale 32nd Edition The Complete Drug Reference (London, The Pharmaceutical Press).

The solid oral compositions may be prepared by conventional methods of blending, filling or tabletting. Repeated blending operations may be used to distribute the active agent throughout those compositions employing large quantities of fillers. Such operations are of course conventional in the art. The tablets may be coated according to methods well known in normal pharmaceutical practice, in particular with an enteric coating.

Oral liquid preparations may be in the form of, for example, emulsions, syrups, or elixirs, or may be presented as a dry product for reconstitution with water or other suitable vehicle before use. Such liquid preparations may contain conventional additives such as suspending agents, for example sorbitol, syrup, methyl cellulose, gelatin, hydroxyethylcellulose, carboxymethylcellulose, aluminium stearate gel, hydrogenated edible fats; emulsifying agents, for example lecithin, sorbitan monooleate, or acacia; non-aqueous vehicles (which may include edible oils), for example almond oil, fractionated coconut oil, oily esters such as esters of glycerine, propylene glycol, or ethyl alcohol; preservatives, for example methyl or propyl p-hydroxybenzoate or sorbic acid; and if desired conventional flavouring or colouring agents.

For parenteral administration, fluid unit dosage forms are prepared utilizing the compound and a sterile vehicle, and, depending on the concentration used, can be either suspended or dissolved in the vehicle. In preparing solutions the compound can be dissolved in water for injection and filter sterilized before filling into a suitable vial or ampoule and sealing. Advantageously, adjuvants such as a local anaesthetic, a preservative and buffering agent can be dissolved in the vehicle. To enhance the stability, the composition can be frozen after filling into the vial and the water removed under vacuum. Parenteral suspensions are prepared in substantially the same manner, except that the Compound (I) suspended in the vehicle instead of being dissolved, and sterilization cannot be accomplished by filtration. The compound can be sterilized by exposure to ethylene oxide before suspending in the sterile vehicle. Advantageously, a surfactant or wetting agent is included in the composition to facilitate uniform distribution of the compound.

Compositions may contain from 0.1% to 99% by weight, preferably from 10-60% by weight, of the active material, depending upon the method of administration.

Composition may, if desired, be in the form of a pack accompanied by written or printed instructions for use.

The compositions are prepared and formulated according to conventional methods, such as those disclosed in standard reference texts, for example the British and US Pharmacopoeias, Remington's Pharmaceutical Sciences (Mack Publishing Co.), Martindale The Complete Drug Reference 32nd Edition and Harry's Cosmeticology (Leonard Hill Books) or the above mentioned publications.

The present invention also provides a pharmaceutical composition comprising an insulin sensitiser, such as Compound (I) especially 2 to 12 mg thereof, an agent used in the treatment of the cardiac conditions associated with diabetes mellitus and a pharmaceutically acceptable carrier therefor, for use as an active therapeutic substance.

In particular, the present invention provides a pharmaceutical composition comprising an insulin sensitiser, such as Compound (I) especially 2 to 12 mg thereof, an agent used in the treatment of the cardiac conditions associated with diabetes mellitus and a pharmaceutically acceptable carrier therefor, for use in the treatment of diabetes mellitus, especially Type II diabetes and the cardiovascular conditions arising from diabetes mellitus.

A range of 2 to 4 mg includes a range of 2.1 to 4, 2.2 to 4, 2.3 to 4, 2.4 to 4, 2.5 to 4, 2.6 to 4, 2.7 to 4, 2.8 to 4, 2.9 to 4 or 3 to 4 mg.

A range of 4 to 8 mg includes a range of 4.1 to 8, 4.2 to 8, 4.3 to 8, 4.4 to 8, 4.5 to 8, 4.6 to 8, 4.7 to 8, 4.8 to 8, 4.9 to 8, 5 to 8, 6 to 8 or 7 to 8 mg.

A range of 8 to 12 mg includes a range of 8.1 to 12, 8.2 to 12, 8.3 to 12, 8.4 to 12, 8.5 to 12, 8.6 to 12, 8.7 to 12, 8.8 to 12, 8.9 to 12, 9 to 12, 10 to 12 or 11 to 12 mg.

No adverse toxicological effects have been established for the compositions or methods of the invention in the above mentioned dosage ranges.

1. A method for the treatment of diabetes mellitus, especially Type II diabetes and the cardiac conditions associated with diabetes mellitus in a mammal such as a human, which method comprises administering an effective non-toxic and pharmaceutically acceptable amount of an insulin sensitiser, such as Compound (I), and an agent used in the treatment of the cardiac conditions associated with diabetes mellitus.

2. A method according to claim 1, wherein the agent used in the treatment of cardiac conditions associated with diabetes mellitus is a beta-blocker, an ACE inhibitor or a diuretic.

3. A method according to claim 1, wherein the agent used in the treatment of cardiac conditions associated with diabetes mellitus is an endothelin antagonist.

4. A method according to any one of claims 1 to 3, wherein the insulin sensitiser is s thiazolidinedione insulin sensitiser.

5. A method according to any one of claims 1 to 4, wherein the thiazolidinedione insulin sensitiser is Compound (I).

6. A method according to any one of claims 1 to 4, wherein the thiazolidinedione insulin sensitiser is selected from the list consisting of: (+)-5-[[4-[(3,4-dihydro-6-hydroxy-2,5,7,8-tetramethyl-2H-1-benzopyran-2-yl)methoxy]phenyl]methyl]-2,4-thiazolidinedione (or troglitazone), 5-[4-[(1-methylcyclohexyl)methoxy]benzyl]thiazolidine-2,4-dione (or ciglitazone), 5-[4-[2-(5-ethylpyridin-2-yl)ethoxy]benzyl]thiazolidine-2,4-dione (or pioglitazone), and 5-[(2-benzyl-2,3-dihydrobenzopyran)-5-ylmethyl)thiazolidine-2,4-dione (or englitazone).

7. A method according to claim 1 or claim 2, wherein the beta-blocker is selected from the list consisting of acebutolol, alprenolol, amosulatol, arotinolol, atenolol, befunolol, betaxolol, bevantolol, bisoprolol, bopindolol, bucindolol, bufetolol, bufuralol, bunitrolol, bupranolol, carazolol, carteolol, carvedilol, celiprolol, chloranolol, dilevalol, epanolol, esmolol, flestolol, indenolol, labetalol, levobunolol, levomoprolol, medroxalol, mepindolol, metipranolol, metoprolol, nadolol, nebivolol, nipradilol, oxprenolol, penbutolol, pindolol, practolol, propranolol, sotalol, talinolol, tertatolol, tilisolol and timolol.

8. A method according to claim 1 or claim 2, wherein the ACE inhibitor blocker is selected from the list consisting of alacepril, benazepril, captopril, ceronapril, cilazepril, delapril, enalapril, enalaprilat, fosinopril, imidapril, libenzapril, lisinopril, moexipril, moveltipril, pentopril, perindopril, quinapril, ramipril, spirapril, temocapril, teprotide, trandolapril and zofenopril.

9. A method according to claim 1 or claim 2, wherein the diuretics is selected from the list consisting of acetazolamide, brinzolamide, dichlorphenamide, dorzolamide, methazolamide, azosemide, bumetanide, ethacrynic acid, etozolin, frusemide, piretanide, torasemide, isosorbide, mannitol, amiloride, canrenoate potassium, canrenone, spironolactone, triamterene, althiazide, bemetizide, bendrofluazide, benzthiazide, buthiazide, chlorothiazide, chlorthalidone, clopamide, cyclopenthiazide, cyclothiazide, epithiazide, hydrochlorothiazide, hydroflumethiazide, indapamide, mebutizide, mefruside, methylclothiazide, meticrane, metolazone, polythiazide, quinethazone, teclothiazide, trichlormethiazide, tripamide and xipamide.

10. A method according to any one of claims 1 to 9 which comprises either co-administration of an insulin sensitiser and the agent used in the treatment of the cardiac conditions associated with diabetes mellitus or the sequential administration thereof.
